(19) United States
(12) Patent Application Publication
(10) Pub. No.: US 2006/0111606 A1
(43) Pub. Date: May 25, 2006

(54) Magnetic therapeutic devices and methods for alleviating pain and assisting healing in humans and animals

(76) Inventor: Frank R. Yee, Montebello, CA (US)

Correspondence Address: Roy L. Anderson, Esq.;LAW OFFICES OF ROY ANDERSON, 1010 North Central Avenue, Glendale, CA, 91202, US

(21) Appl. No.: 10/993,939

(22) Filed: Nov. 19, 2004

(51) Int. Cl. A61N 2/08

(52) U.S. Cl. 600/15

(57) ABSTRACT

A magnetic therapeutic device having one or more flexible strands (such as monofilament nylon line) detachably connectable at a first end with a first end magnetic clasp and a second end with a second end magnetic clasp while the strands hold magnetized hematite beads (which substantially surround the strands) and, optionally, decorative beads (which may be made of a natural stone), between the first and the second ends so that a closed loop with a continuous magnetic field (which can have a strength of at least approximately 50,000 gauss) is formed when the first and the second magnetic clasp are joined together. When multiple strands are included in a single device, multiple magnetic clasps and spacers (which may be magnetized hematite beads) hold the strands together. The therapeutic can be a necklace or a bracelet worn on a wrist or ankle by humans, or a neck collar or leg wrap worn by animals.

### FIELD OF THE INVENTION

The present invention is in the field of magnetic therapeutic devices and methods that use magnetism to assist in healing and alleviating pain in humans and animals.

### BACKGROUND OF THE INVENTION

It is known that magnetism is effective for therapy or removal of aches, as is described in U.S. Pat. No. 5,807,233 to Sakuma et al. In this patent, a faint magnetism of 2-20 gauss is used to alleviate pain. To provide this faint field, this patent teaches that magnetic fibers can be arranged in a belt or pouch.

More recently, magnetic therapeutic devices for application to a target pain or injury area are described in U.S. Pat. No. 6,632,168 to Roberts et al., the disclosure of which is incorporated herein by reference. In the background of the invention of this patent, scientific studies regarding magnetism and its power to reduce pain or assist in healing are described. The patent goes on to disclose magnetic therapeutic devices for application to a target pain or injury area under a user's skin, as well as methods for using such devices. The devices disclosed use a magnet or magnetic material that ends up being located near a desired area of the body by a hook like member or a patch. In contrast to U.S. Pat. No. 5,807,233 to Sakuma et al., this patent describes use of a significant magnetic field greater than about 12,000 gauss (see column 5, line 53).

Additional magnetic therapeutic devices are described in U.S. Patent Application Publication No. US 2003/0004393 A1 to Ewing et al., the disclosure of which is also incorporated herein by reference. This publication describes devices having a magnetic source that can be inserted into a body cavity and devices for applying a magnetic field to the lower temporal/mandibular region of a human head.

It has also been suggested that a neodymium magnet can be included inside of a plastic tube, and worn as a piece of jewelry, to thereby achieve therapeutic benefits, and such jewelry is described in U.S. Patent Application Publication US 2004/0092788 A1 to Kaufman et al.

The present invention goes beyond the teachings of the foregoing references and discloses new therapeutic devices that are efficient, long-lasting and economical, which can also be made into fashionable jewelry. In addition, the present invention discloses how such devices can be used with animals, such as dogs, cats and horses, to name a few.

### SUMMARY OF THE INVENTION

The present invention is generally directed to a magnetic therapeutic device having one or more flexible strands (such as monofilament nylon line) detachably connectable at a first end with a first end magnetic clasp and a second end with a second end magnetic clasp while the strands hold magnetized hematite beads (which substantially surround the strands) and, optionally, decorative beads (which may be made of a natural stone), between the first and the second ends so that a closed loop with a continuous magnetic field (which can have a strength of at least approximately 50,000 gauss) is formed when the first and the second magnetic clasp are joined together. When multiple strands are included in a single device, multiple magnetic clasps and spacers (which may be magnetized hematite beads) hold the strands together. The therapeutic device can be a necklace or a bracelet worn on a wrist or ankle by humans, or a neck collar or leg wrap worn by animals.

In a first, separate group of aspects of the present invention, a method for alleviating a pain in a portion of a body in humans and animals is described in which one or more magnetic magnetic therapeutic devices having one or more strands are worn about a body region until the pain is reduced. When the magnetic therapeutic devices are used with animals, an additional wrap can be used, and such wrap can be treated with pepper when it is used on legs of horses.

Accordingly, it is a primary object of the present invention to provide improved therapeutic devices and methods that use magnetism to assist in healing and alleviating pain in humans and animals.

This and further objects and advantages will be apparent to those skilled in the art in connection with the drawings and the detailed description of the preferred embodiment set forth below.

### BRIEF DESCRIPTION OF THE DRAWINGS

FIG. 1 is a drawing showing a magnetic therapeutic device according to the present invention in a detached state.

FIG. 2 is a drawing showing a second magnetic therapeutic device according to the present invention in a connected state.

FIG. 3 shows opposing magnetic clasps used in a magnetic therapeutic device according to the present invention.

FIG. 4 shows a variety of shapes of hematite and decorative beads useful in a magnetic therapeutic device according to the present invention.

FIG. 5 is a top planar view of a magnetic clasp affixed to a strand used in a magnetic therapeutic device according to the present invention.

FIG. 6 is a drawing showing a two-stranded magnetic therapeutic device according to the present invention in a detached state.

### DETAILED DESCRIPTION OF THE INVENTION

The present invention provides a magnetic therapeutic device for use with humans or domesticated animals, such as cats, dogs and horses. The magnetic therapeutic device is worn around a region of a body, preferably near the area of the body that is experiencing pain or is healing. In contrast to prior therapeutic devices, the present invention uses two magnetic clasps and magnetized hematite beads held on a flexible stand to form a closed loop with a constant continuous magnetic field in which a magnetic field is present throughout the closed loop and is not broken by an insulator which interrupts the magnetic field. Because the closed loop has a continuous magnetic field, the therapeutic devices do not become demagnetized, or lose their magnetic field as readily, as prior therapeutic devices, thus increasing effectiveness and usefulness over time.

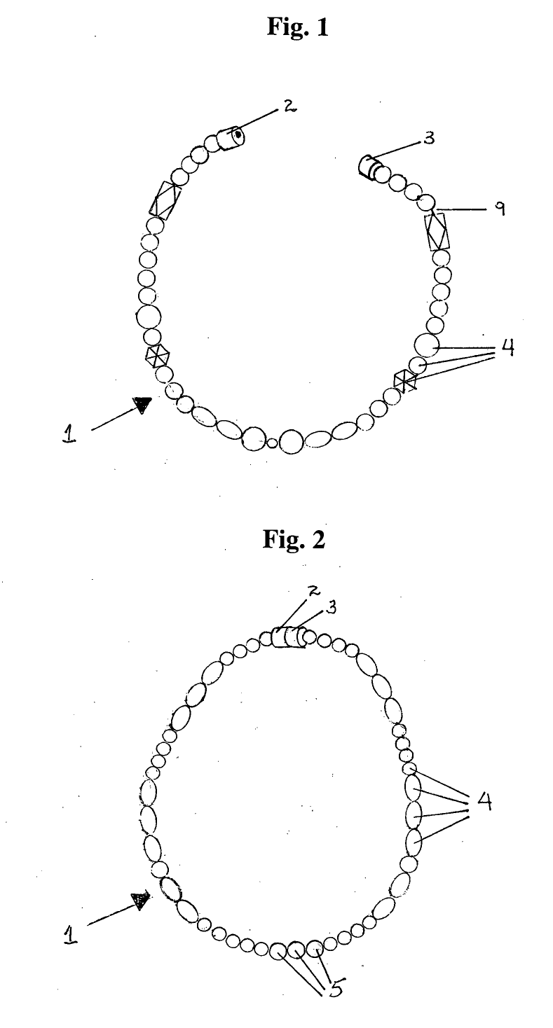

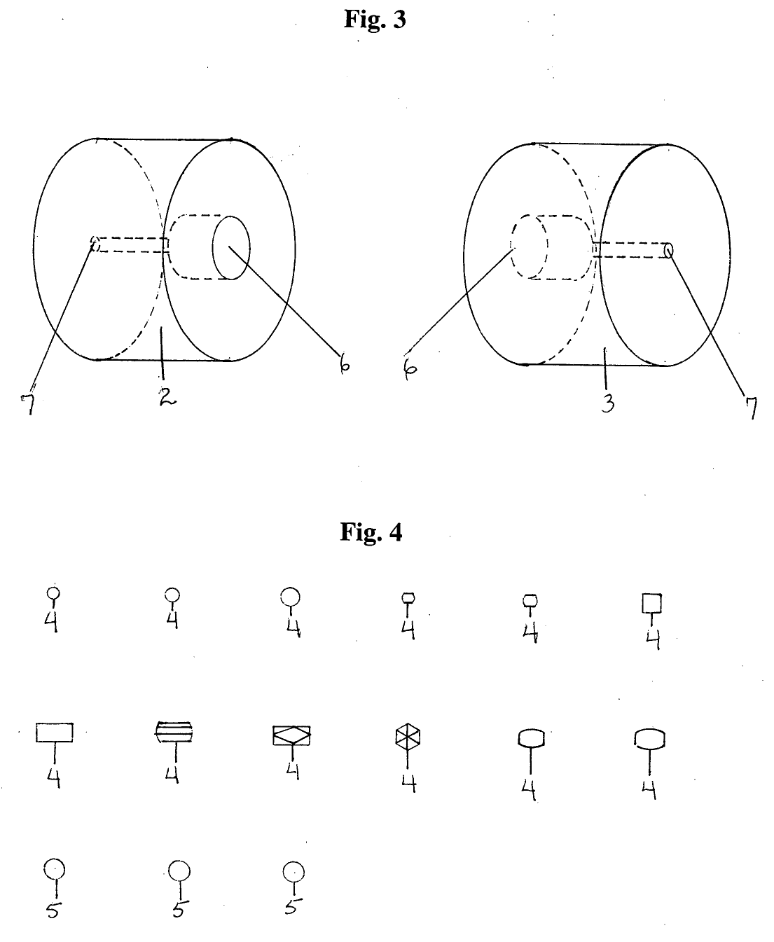

An especially preferred embodiment, shown generally as 1 in FIG. 1, fashions the therapeutic device into a necklace or bracelet (for either a human wrist or ankle) having two ends on which two magnet clasps, 2 and 3, are affixed. Magnetic clasps 2 and 3 are affixed to a flexible strand 9 and multiple hematite beads 4, which have been magnetized, are threaded onto flexible strand 9 and held between magnet clasps 2 and 3 once the magnet clasp are affixed in place at their respective ends.

The magnetic clasps used in the present invention are, in an especially preferred embodiment, a pair of magnetic cylindrical ends with a hole in the center of the clasps so they will hold a monofilament line. The magnetic clasps have N pole and S pole polarity therefore creating the ability to connect to each other. A single, one hole, magnetic clasp is 6 mm×5 mm or 6 mm×6 mm and has a 3500 gauss rating, so a single pair of clasps would be around 7000 gauss. A Double, two hole, magnetic (rectangular shape) clasp is 6 mm×6 mm×12 mm and the gauss rating is 4500. With a single pair of double clasps it would be around 9000 gauss.

Flexible strand 9 can be a monofilament line or something else, as long as it does not prevent the therapeutic device 1 from having a constant, uninterrupted magnetic field when it is configured in a closed loop (due to the North/South alignment of the magnetic clasps and the magnetized hematite beads). The flexible strand used in an especially preferred embodiment of the present invention is a nylon-alloy monofilament line (such as a fishing line). The Polymer Chemist defines nylons as synthetic linear polymers containing polymide linkages. The arrangement of these polyamide linkages in space, their relationship to one another and proximity to one another from molecule to molecule determine such properties as melting point, crystallinity, strength, stiffness, etc. From a practical standpoint, nylons are a broad class of plastics that have the ability to be melted and extruded into monofilament that can be stretched or oriented several fold to give strong flexible fibers. All nylons are composed of carbon, hydrogen, oxygen and nitrogen. The special arrangement of these atoms or elements within the molecules of “Nylon” account for the properties of this unique type of plastic (or polymer as the chemist refers to them). The strength of the line used in an especially preferred embodiment will vary from 30# to 80# test and the diameter of the line will vary in order to fit in the size of the hole in the mineral bead or hematite and to add strength. The properties of the nylon-alloy monofilament line enhance the magnetic field.

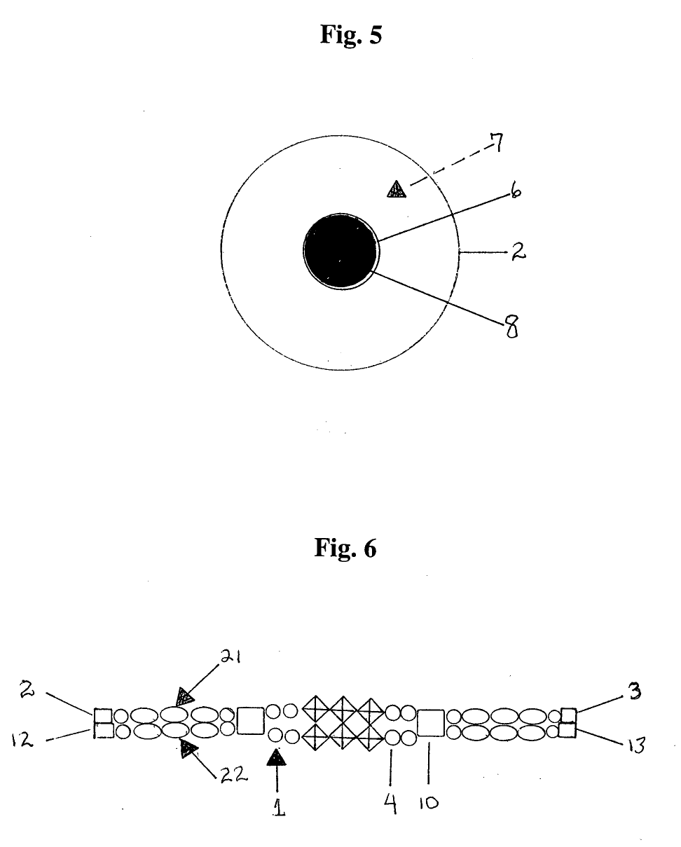

Magnet clasps 2 and 3 have a center hole 7 running through them and a cup- or ball-shaped indentation 6 in their ends. A first magnetic clasp is affixed to flexible strand 9 by threading flexible strand 9 through center hole 7 and having it extend outwardly a short distance from indentation 6. Next, flexible strand 9 is heated, in response to which it melts until a ball-like end 8 (see FIG. 5) with a diameter larger than flexible strand 9 is formed and then held within indentation 6 in a process similar to that which is described in U.S. Pat. No. 3,778,871, the disclosure of which is incorporated herein by reference. This process will seat the ball into the concave portion of the clasp, thus preventing the withdrawal of the line from the clasp. In addition, the melted ball takes the shape of a symmetric and spherical surface inside the clasp. Once one magnetic clasp is attached, hematite beads 4 and decorative beads 5 (if used) are threaded onto flexible strand 9 until a desired length is achieved. At this point, the remaining end of flexible strand 9 is threaded through center hole 7 of the second magnet, subjected to heat, and then the resultant ball is burned into indentation 6 of the second magnet. It is especially preferred that hematite beads 4 and decorative beads 5 (if used) substantially surround the strand between the first and the second ends so as to avoid gaps between beads and clasps, to the extent possible, to create an uninterrupted series of beads and clasps; in any event, there must be sufficient hematite beads 4, which are appropriately sized and spaced, so that therapeutic device 1, when it is in a connected state forming a closed loop, has a continuous magnetic field in the closed loop. Gaps between beads can be eliminated by pulling the beads on a strand toward the first magnetic clasp before the second magnetic clasp is affixed to the strand, and then placing the second magnetic clasp on the strand maintaining contact between all the beads, and then burning the second clasp so that all of the beads are in continuous contact with each other and the two clasps.

Hematite is a natural mineral, which can be made into a shiny, black, magnetized bead. The magnetized beads vary in size and shapes. Commonly available sizes are 2 mm to 24 mm, while commonly available shapes are round, drum, tear drop, faceted, cubes, rectangle, hexagonal, 18 facets, twist, 6 facets, rondelle, double cone, spacers, rice, etc. The natural property of magnetized hematite creates an uninterrupted magnetic field. One magnetized hematite stone might have a magnetic field from as low as 50 gauss to as high as 1500 gauss.

In an alternative preferred embodiment, shown in FIG. 2, one or more decorative beads 5 made of a “natural stone”, such as jade, jasper, turquoise or pearl, are also threaded onto flexible strand 9 between magnet clasps 2 and 3. The decorative beads can themselves be selected for therapeutic purposes, such as is described in U.S. Patent Publication No. US 2004/0197419 A1 to Cole et al., the disclosure of which is incorporated herein by reference. The “natural stone” beads may also be fiber optics beads, which can be very colorful, since fiber optics beads can be excellent for conducting magnetism. Accordingly, while “natural stone” beads can take many forms, and be made of many materials, not all of which might be considered a “stone,” the key is that the decorative beads do not act as an insulator to break the magnetic field formed when the magnet clasps of a therapeutic device are fixed together to thereby form a closed loop. So, according this definition, many synthetic plastic beads will not qualify as “natural stone” because they will interrupt the magnetic field, creating a gap in the closed loop, thus preventing a continuous magnetic field from being formed in the closed loop in which they are used.

Hematite beads 4 and decorative beads 5 can be formed in many different shapes and sizes, and do not need to be round or shaped in a ball; instead, they can take on any shape, even an ornamental or fanciful shape or design, so long as they can be threaded onto flexible strand 9. FIG. 4 is illustrative, but not limiting, of some examples of common bead shapes and sizes that can conveniently be used in the present invention.

In a third preferred embodiment, shown in FIG. 6, two strands, 21 and 21, having two pairs of magnetic clasps, 2 & 3 and 12 & 13, are held together in part by magnetized hematite spacers 10 and by attraction of magnetic clasps of like polarity (2 & 12 and 3 &13). The spacers 10 physically hold the two strands together because the spacers have two holes in a single physical bead and the flexible strands of both of the strands are threaded through each spacer.

In all of the preferred embodiments, the length of the single or double strand devices will depend on the location where it is to be worn, i.e.: neck, wrist, or ankle in humans, or neck or leg of an animal, and individual devices can be fitted to individual human dimensions. The beads and spacers are threaded on the flexible strand(s) insuring that all are in polarity. Each of the spacers 10 and magnetized hematite stones 4 will be threaded with N pole and S pole arrangement to lie alternately in a circumferential direction to form a magnetized surface. The effect of all the magnetized hematite beads, natural stones (if used), magnetized clasp, and fluorocarbon nylon line touching each other and the strength (gauss) creates an additive effect in which the whole is greater than the sum of its parts. If a stronger magnetic field is desired, larger magnetized hematite beads will be used, for quicker pain relief. An average 7-inch single strand bracelet will have 35-40 magnetized hematite stones (with a cumulative magnetic field of approximately 60,000 gauss) and one pair of magnetized clasps (each clasp having a magnetic field of 3500 gauss) so that the total magnetic field for the 7-inch single strand bracelet is approximately 67,000, whereas a double stand device of the same dimensions will have a magnetic field of 134,000 gauss.

Thus, in sum, the magnetic therapeutic device of the present invention provides a magnetic chain, which provides for the ability to attach to itself. The magnetic chain, band or strand can be worn in humans on a neck as a necklace, or on a wrist as a bracelet, or on an ankle as an anklet and, if it is long enough, around a waist. In animals, the magnetic chain can be worn as a neck collar, or be adapted to be worn on a leg with an additional wrap to keep it in place. This magnetic jewelry is adapted for both ornamental and therapeutic use. The magnetic chain is made up from two magnetic claps, nylon-alloy monofilament line, and magnetized hematite. This jewelry is used to create a continuous magnetic field when humans and animals wear it to increase blood circulation and carry more oxygen to the region, decrease inflammation, and relieve pain. The magnetic field may affect pain receptors in the painful area, eliciting a slight anesthetic affect, or the magnetic field might be transmitted via blood vessels to the brain, which then releases endorphins, chemicals that act as natural pain relievers. Since the device has a constant and contiguous magnetic field, the hematite beads do not as readily become demagnetized over time.

The present invention will now be explained in further detail by setting forth some case histories of humans who have worn a magnetic therapeutic device in accordance with the present invention. In each case, while the magnetic field strength of the device was not individually measured, it would have been comparable to the strength, accounting for length and the number of strands, with the magnetic 7 inch device already described having an approximate magnetic field strength of 67,000 gauss. The case histories are based upon the statements of the individuals who wore the devices. The following case histories are meant to be illustrative, but not limiting, of the invention.

1. A 54-year-old lady broke her little finger on her right hand when she was 14 years old. She could not straighten it. A double strand bracelet was put on her right wrist. In 5 minutes she was able to straighten her finger, without pain.

2. A 35-year-old man had a headache. He put on a 17″ necklace. The area where the necklace was touching the skin got a little warmer than other parts of his body. His headache started to decrease in 2 minutes. His headache was completely gone in 20 minutes.

3. A 40-year-old lady had menstrual cramps at 2:00 a.m. She couldn't sleep and had severe cramping, she put her necklace on, and the cramping went away in 2 minutes. Now, she will not take her necklace off and she will not even let her girlfriend use it.

4. A 15-year-old boy was sitting in the back of his van. He was getting motion sickness. His mother gave him her hematite necklace. His motion sickness went away in 5 minutes.

5. A 33-year-old man didn't have any pain, but he had insomnia. He put on a hematite necklace. That night he slept like a baby.

6. A 28-year-old lady got into a bad car accident. She broke 2 legs and had pain in her legs and back. She put on 2 anklets, 2 bracelets and a necklace. Now, she does not have any pain and her doctor said she is healing 200% faster.

7. A 37-year old had bad carpal tunnel in both hands and her fore arms hurt. She put on two bracelets. Her pain went away in five minutes.

8. A 66-year-old man had a stroke and had real bad arthritis in his left hand. He could not open his hand. He put on a bracelet and necklace. In 3 minutes he opened his hand about ½ in. His pain in his neck and shoulder had decreased. In 1 hour his pain in his neck and shoulders decreased by 50%. The next day his hand was completely open.

9. A 55-year-old lady has heel spurs in her left foot and was in deep pain. She put an anklet on and in 5 minutes the pain went away.

10. A 40-year-old lady has pain in her right hip joint and back pain. She put on a bracelet and necklace. Her pain went away in 10 minutes.

As is apparent from the above ten case histories, the devices do not have to be worn over the area where the pain occurs, and multiple devices in different locations can be combined together, thus creating larger magnetic fields applied to the body. Also, the devices are useful for alleviating the symptoms or pain of a variety of ailments.

Based upon the experiences of the persons in these case histories and others, it has been determined that people wearing a magnetic therapeutic device according to the present invention may experience certain common symptoms. For example, a slight tingling may be noticed in the region where a hematite is, and the region may feel like it is getting warmer. Also, it may feel like the hematite is still there, even when the device is taken off. If a person does not have a particular pain, wearing the device can result in a deeper sleep, with good dreams, and waking up more refreshed, and such persons also notice an increase in energy level; the same symptoms are obtained by persons with pain, plus less pain and irritability. Finally, it has been noticed that a very small percentage of people have a reaction to metal on the skin, which can take the form of a scratchy, itchy feeling, or even a rash; to remedy this, a Teflon® coated clasp can be used to replace a silver looking magnetic clasp.

The invention will now be explained in further detail by setting forth some case histories of animals who have worn a magnetic therapeutic device in accordance with the present invention. In each case, while the magnetic field strength of the device was not individually measured, it would have been comparable to the strength, accounting for length and the number of strands, with the magnetic 7 inch device already described having an approximate magnetic field strength of 67,000 gauss. The case histories are based upon the statements of individuals who owned the animals. The following case histories are meant to be illustrative, but not limiting, of the invention.

1. A 38-year-old man has a 10-year-old German Shepard dog, which has arthritis in the dogs left hip joint causing limping. A 23-inch necklace was put on the dog's neck. In 6 hours the dog was not limping. After 24 hours, the dog was playing as if it were 2-years-old.

2. A 32-year-old lady has 8-year-old Siamese cat that was slow and sluggish. A 10-inch single strand necklace was attached. After 12 hours the cat was very active, alert, and playing with the other cats.

3. A 9-year-old poodle would not climb the stairs due to the pain in its front legs. A 10-inch necklace was attached. After 8 hours the dog was running up and down the stairs and jumping on the furniture.

4. A small dog was lazy, slept 20 hours a day. A 10-inch necklace was attached to the neck. After 20 minutes, the dog was walking around looking alert. After 24 hours the dog was romping and playing with the other dogs and the appetite came back to normal.

When a magnetic therapeutic device in accordance with the present invention is worn by an animal, it has been found that it is best to use a double stranded device because it is less likely that the animal will be able to remove it. Also, to avoid having the device coming off by a scratching action, especially in the case of a neck collar, an additional wrap can be placed around the device, or a tied ribbon next to the clasp can be used to further secure the device. And, in the case of horses, when a device has been held on a horse's leg with an additional wrap (such as an Ace bandage), it has been found that the horses will try to bite the wrap and devices to remove them. To remedy this situation, it has been found that inclusion of a pepper (e.g., cayenne pepper or Tabasco® sauce) on the wrap will deter such action by the horse.

While the invention has been described herein with reference to certain preferred embodiments, those embodiments have been presented by way of example only, and not to limit the scope of the invention. Additional embodiments thereof will be obvious to those skilled in the art having the benefit of this detailed description. For example, just as one additional strand has been added in the double stranded magnetic therapeutic device shown in FIG. 6, even further strands could be added in a similar fashion. Further modifications are also possible in alternative embodiments without departing from the inventive concept.

Accordingly, it will be apparent to those skilled in the art that still further changes and modifications in the actual concepts described herein can readily be made without departing from the spirit and scope of the disclosed inventions as defined by the following claims.

What is claimed is:

1. A magnetic therapeutic device, comprising:
a flexible strand detachably connectable at a first end with a first magnetic clasp and a second end with a second magnetic clasp, and
a plurality of magnetized hematite beads held by the strand between the first and the second ends;
wherein a closed loop is formed when the first and the second magnetic clasp are joined together; and
wherein the closed loop creates a continuous magnetic field.

2. The magnetic therapeutic device of claim 1, wherein the uninterrupted magnetic field has a strength of at least approximately 50,000 gauss.

3. The magnetic therapeutic device of claim 1, further comprising one or more decorative beads.

4. The magnetic therapeutic device of claim 3, wherein the one or more decorative beads are comprised of a natural stone.

5. The magnetic therapeutic device of claim 5, wherein the magnetic therapeutic devices is a necklace.

6. The magnetic therapeutic device of claim 1, wherein the magnetic therapeutic device is a collar for an animal.

7. The magnetic therapeutic device of claim 1, wherein the magnetic therapeutic device is a bracelet.

8. The magnetic therapeutic device of claim 1, wherein the plurality of magnetized hematite beads substantially surround the strand between the first and the second ends.

9. The magnetic therapeutic device of claim 3, wherein the plurality of magnetized hematite beads and the one or more decorative beads substantially surround the strand between the first and the second ends.

10. The magnetic therapeutic device of claim 1, wherein the strand is a monofilament nylon line and the plurality of magnetized hematite beads are threaded onto the line.

11. The magnetic therapeutic device of claim 1, further comprising:
a second flexible strand detachably connectable at the first end with a third magnetic clasp and the second end with a fourth magnetic clasp;
a second plurality of magnetized hematite beads held by the second strand between the first and the second ends; and
one or more spacers held by both the first and the second strands between the first and the second ends.

12. A magnetic therapeutic device, comprising:
two or more flexible strands detachably connectable between a first end and a second end, each of the plurality of flexible strands having a first magnetic clasp attached at the first end and a second magnetic clasp attached at the second end,
two or more pluralities of magnetized hematite beads held by the two or more strands between the first and the second ends; and
a plurality of spacers held by the two or more flexible strands between the first and second ends;
wherein a closed loop is formed when the first and the second magnetic clasps of the two or more flexible strands are joined together; and
wherein the closed loop creates a continuous magnetic field.

13. The magnetic therapeutic device of claim 12, wherein the plurality of spacers are comprised of magnetized hematite.

14. A method for alleviating a pain in a portion of a body, comprising:
wearing a magnetic therapeutic device having a closed loop about a body region until the pain is reduced;
wherein the closed loop is comprised of:
a flexible strand detachably connectable at a first end with a first magnetic clasp and a second end with a second magnetic clasp; and
a plurality of magnetized hematite beads held by the strand between the first and the second ends;
wherein the closed loop is formed when the first and the second magnetic clasps are joined together; and
wherein the closed loop creates a continuous magnetic field.

15. The method of claim 14, wherein the body is a human body.

16. The method of claim 14, wherein the body is an animal body.

17. The method of claim 16, wherein the body is a horse body.

18. The method of claim 17, wherein the closed loop is wrapped around a leg of the horse and then covered by a wrap material treated with a pepper.

19. The method of claim 14, wherein the closed loop is further comprised of
a second flexible strand detachably connectable at the first end with a third magnetic clasp and the second end with a fourth magnetic clasp;
a second plurality of magnetized hematite beads held by the second strand between the first and the second ends; and
one or more spacers held by both the first and the second strands between the first and the second ends.

20. The method of claim 14, comprising the further step of:
wearing a second magnetic therapeutic device having a second closed loop about a second body region until the pain is reduced, wherein the second closed loop is comprised of:
a second flexible strand detachably connectable at a first second strand end with a first second strand magnetic clasp and a second second strand end with a second second strand magnetic clasp; and
a plurality of second strand magnetized hematite beads held by the second strand between its first and second ends;
wherein the second closed loop is formed when the first and the second magnetic clasps of the second strand are joined together; and
wherein the second closed loop creates a second continuous magnetic field.
